(19) United States
(12) Patent Application Publication
(10) Pub. No.: US 2014/0012305 A1
(43) Pub. Date: Jan. 9, 2014

(54) COLPOTOMY CUP-LIKE STRUCTURE AND INTRAUTERINE MANIPULATOR INCLUDING SAME

(71) Applicants: Jeremy C. Horton, Salt Lake City, UT (US); Richard A. Dixon, Bountiful, UT (US)

(72) Inventors: Jeremy C. Horton, Salt Lake City, UT (US); Richard A. Dixon, Bountiful, UT (US)

(73) Assignee: Clinical Innovations, LLC, Murray, UT (US)

(21) Appl. No.: 13/541,956

(22) Filed: Jul. 5, 2012

(51) Int. Cl. A61M 29/00

(52) U.S. Cl. 606/193

(57) ABSTRACT

A cup-like structure for engaging a cervix of a patient includes a rim, and includes a base defining an aperture through which one or more tubular members of a uterine manipulator may extend into the uterus. The cup-like structure is made from one or more of a polyphthalamide (PPA) material and a polyetheretherketone (PEEK) material. A uterine manipulator including a cup-like structure is also disclosed.

### TECHNICAL FIELD

The present disclosure relates to medical instrumentation for manipulating a position of a uterus for better visualization and surgical access, and more specifically, to a cup-like structure for engaging the cervix of a patient and a uterine manipulator equipped with the cup-like structure.

### BACKGROUND

Uterine manipulators, including intrauterine manipulators, are commonly used by practitioners for all laparoscopies involving the female pelvic organs (uterus, tubes, ovaries) when a uterus is present, as surgery performed without the use of a uterine manipulator is more dangerous and can be more time consuming. Examples of laparoscopic procedures in which a uterine manipulator has substantial utility include: tubal ligations for sterilization; diagnostic laparoscopies for evaluating pelvic pain and infertility; treatment of endometriosis; removal of pelvic scars (adhesions) involving the uterus, fallopian tubes and ovaries; treatment of ectopic pregnancy; removal of uterine fibroids; removal of ovarian cysts; removal of ovaries; tubal repair; laparoscopic hysterectomy; laparoscopic repair of pelvic bowel or bladder; sampling of pelvic lymph nodes; “tying up” the bladder to prevent urine loss; and biopsy of pelvic masses. Intrauterine manipulators are also employed as conduits for the delivery of dye into the uterus when the physician wishes to obtain a picture of the uterus (hysterosalpingogram). One state of the art manipulator 12, as shown in FIG. 1, and as offered by the assignee of the present invention as the CLEARVIEW® uterine manipulator, is an instrument 12 which includes a distal portion 14 coupled to a rigid member 16, such as an insertion rod. The instrument 12 is inserted through the vagina and attaches in a fixed manner to the uterus while a portion of the instrument 12, including a handle 18 with a control mechanism 20 thereon, protrudes from the vagina. The instrument 12 may include a vaginal occluder (not shown) for sealing the patient's abdominal cavity with carbon dioxide (CO₂). The instrument 12 may be held in place by in part by a cup-like structure 22 designed to engage the patient's cervix. More specifically, the cup-like structure 22 has a rim 24 sized to envelop the anterior and posterior formix. A base 26 of the cup-like structure 22 abuts the cervix and has an aperture therein aligned with the cervical os and allows a tip section 28 to extend therethrough and into the uterus. The tip section 28 may include a balloon 30, a supple tip 32, and one or more tubes 34, 36 enabling the physician to inflate the balloon 30, to inject dye into the uterus through a port 37, or both. The tip section 28 may be sized to enter the uterus through the cervical os with minimal, if any, dilation of the cervix.

Once inside the uterus, the balloon 30 may be inflated to engage the interior uterine wall in a manner wherein the uterus is non-traumatically gripped between the tip section 28 and the cup-like structure 22 of the instrument 12. Cup-like structure 22 is commonly termed a “colpotomy cup,” and such terminology, as well as the term “cup,” may be used herein for convenience and not limitation of the design or configuration of the referenced structure. Once the uterus is gripped between the tip section 28 and the cup-like structure 22, manipulation of the uterus is accomplished by rotating the distal portion 14 about a pivot point 38 proximate the cup-like structure 22. Typically, the instrument 12 is inserted in an orientation such that the rotation of the distal portion 14 about the pivot point 38 occurs in a front-to-back manner with respect to the anterior and posterior of the patient. The rotation of the distal portion 14 may be manipulated by the control mechanism 20, which, as depicted, is a rotatable knob 40 located at or near the handle 18. In such a manner, the instrument 12 allows the physician to manipulate the orientation of the uterus as desired. For example, if the physician wishes to rotate the uterus into an anteverted position, she may rotate the knob 40 in a clockwise direction. To rotate the uterus into a retroverted position, the physician may rotate the knob 40 in a counterclockwise direction. Lateral (left-to-right) rotation of the uterus can also be accomplished by manipulating the rigid member 16 or by orienting the instrument 12 during insertion wherein rotation of the distal portion 14 about the pivot point 38 occurs laterally with respect to the patient.

The cup-like structure 22 may include a circumferential protrusion 42 outwardly radially extending therefrom at or near the rim 24. When the cup-like structure 22 has engaged the cervix as previously described, the physician can visually locate the protrusion 42 by identifying a corresponding deformation on the outer surface of the vaginal formices. In laparoscopic procedures, the physician views the uterus from a trocar-mounted camera inserted into the abdominal cavity through the abdominal wall. During a colpotomy procedure, the physician uses a scalpel to make an incision in the vaginal formices at or near the protrusion 42. In this manner, the protrusion 42, or other portion of the cup-like structure 22, such as the rim 24, may act as a backing, or “back-stop,” for the scalpel. Several types of scalpels are commonly used in colpotomy procedures, including electrosurgical scalpels (e.g., scalpels using a radiofrequency oscillating electrical current), harmonic scalpels (e.g., ultrasonic scalpels) and laser scalpels (e.g., CO₂ or YAG lasers), and are understood by persons of ordinary skill in the art.

A major drawback to conventional cup-like structures 22 currently available is that the cups must be manufactured to accommodate use of one or two, but not all, of an electrosurgical scalpel, a harmonic scalpel, or a laser scalpel. For example, metal cups are commonly used with harmonic scalpels because the metal materials have a high melting temperature and can withstand the intense thermal loads generated by high-frequency vibration, on the order of 55,500 kHz, employed by harmonic scalpels. However, metal cups cannot be used with electrosurgical scalpels because an electrically conductive metal cup will, among other things, short out an electrosurgical scalpel if the scalpel contacts the cup. Similarly, metal cups may not be indicated for use with laser scalpels due to undue heat absorption from contact with the laser beam, particularly with the relatively high power beam of a CO₂ laser. Conversely, electrically insulating cups, such as plastic or polymeric cups, are commonly used with electrosurgical scalpels because they do not pose a threat to the electronic functionality of such scalpels if the scalpel contacts the cup; however, conventional plastic or polymeric cups cannot be used with harmonic scalpels because the cup will, among other things, warp, melt (often with jagged melt edges), burn, disassociate into harmful particulate fragments, and/or emit undesirable gasses if used therewith. Similar issues arise with the use of conventional plastic or polymeric cups in conjunction with laser scalpels. Ceramic cups have also been found to have shortcomings when used with harmonic scalpels. While ceramic materials may possess high melting temperatures generally beneficial for use with heat-intensive cutting devices, noncompliant ceramic material risks fracturing a harmonic scalpel, or becoming fractured itself, if the scalpel contacts the cup, producing harmful particulates that may be painful and pose the risk of infection for the patient.

Since various sizes of colpotomy cups must be available for use, commonly multiple different-sized cups are supplied. As materials of conventional colpotomy cups, as noted above, are not suitable for use with both electrosurgical and harmonic scalpels, this necessitates an even greater multiple of cups, two of each size of different materials, respectively suitable for use with each type of scalpel. Consequently, there is undesirable duplication of cups.

### BRIEF SUMMARY

This summary is provided to introduce a selection of concepts in a simplified form. These concepts are described in further detail in the detailed description of embodiments of the disclosure below. This summary is not intended to identify key features or essential features of the claimed subject matter, nor is it intended to be used to limit the scope of the claimed subject matter.

In view of the above-referenced shortcomings in the current state of colpotomy cup offerings, the inventors have devised a colpotomy cup for use with a uterine manipulator and that may be used with electrosurgical, harmonic and laser scalpels. Such a colpotomy cup may reduce costs and offer physicians a greater number of options when selecting instrumentation for performing a uterine laparoscopy. With the increasing pressure for cost containment in medical treatment and the increasing popularity of laparoscopic gynecological procedures moving to outpatient clinics and GYN offices, the potential for a more versatile colpotomy cup is very attractive to the medical profession.

In some embodiments, the present disclosure includes an intrauterine manipulator assembly including a distal portion in communication with a handle. The distal portion includes a balloon for engaging the inside of patient's uterine wall. The distal portion also includes a cup-like structure for engaging the patient's cervix. The cup-like structure includes a rim, and a base defining an aperture through which one or more tubular members may extend. The base is located proximate the balloon. The cup-like structure consists essentially of a polyphthalamide (PPA) material or polyetheretherketone (PEEK) material.

In additional embodiments, the present disclosure includes a cup-like structure for engaging a cervix of a patient and includes a rim configured to surround at least a portion of the anterior and posterior vaginal formix of the patient. The cup-like structure also includes a base defining an aperture therein. At least one of the rim and the base consist essentially of one of a polyphthalamide (PPA) material or a polyetheretherketone (PEEK) material.

### BRIEF DESCRIPTION OF THE DRAWINGS

While the specification concludes with claims particularly pointing out and distinctly claiming what are regarded as embodiments of the invention, the advantages of embodiments of the disclosure may be more readily ascertained from the description of certain examples of embodiments of the disclosure when read in conjunction with the accompanying drawings, in which:

FIG. 1 is a side view of a uterine manipulator having a cup-like structure for engaging the cervix of a patient;

FIG. 2 is a perspective view of the cup-like structure for use with a uterine manipulator;

FIG. 3 is a side view of the cup-like structure of FIG. 2;

FIG. 4 is a top view of the cup-like structure of FIGS. 2 and 3; and

FIG. 5 is a cross-sectional view of the cup-like structure of FIG. 4 taken along section line 5-5.

### DETAILED DESCRIPTION

The illustrations presented herein are not meant to be actual views of any particular instrument, structure, or device, but are merely idealized representations that are used to describe embodiments of the disclosure.

As used herein, the terms “cup” and “colpotomy cup” mean and include any cup-like structure configured to engage and envelop at least a portion of a cervix and which may generally be shaped to correspond with the shape of a cervix, and are not limited to use in any specific type of medical procedure.

As used herein to describe a behavior or characteristic, observed or inherent, of a cup-like structure or of a material used in a colpotomy cup (as defined herein), the term “substantially does not” means that the cup-like structure or the material does not exhibit the referenced behavior or possess the referenced characteristic to an extent or degree to render the cup-like structure or material unsatisfactory for use, respectively, as a colpotomy cup or in a colpotomy cup.

By way of non-limiting example, when referring to a cup-like structure, the term “substantially does not disassociate particulate fragments” means that the cup-like structure does not disassociate particulate fragments to an extent or degree to render the cup-like structure unsatisfactory for use as a colpotomy cup.

By way of another non-limiting example, when referring to a cup-like structure, the term “substantially does not melt or burn” means that the cup-like structure does not melt or burn to an extent or degree to render the cup-like structure unsatisfactory for use as a colpotomy cup.

By way of yet another non-limiting example, when referring to a cup-like structure, the term “substantially does not form jagged melt edges” means that the cup-like structure does not form jagged melt edges to an extent or degree to render the cup-like structure unsatisfactory for use as a colpotomy cup.

By way of an additional non-limiting example, when referring to a cup-like structure, the term “substantially does not emit smoke or gas” means that the cup-like structure does not emit smoke or gas to an extent or degree to render the cup-like structure unsatisfactory for use as a colpotomy cup.

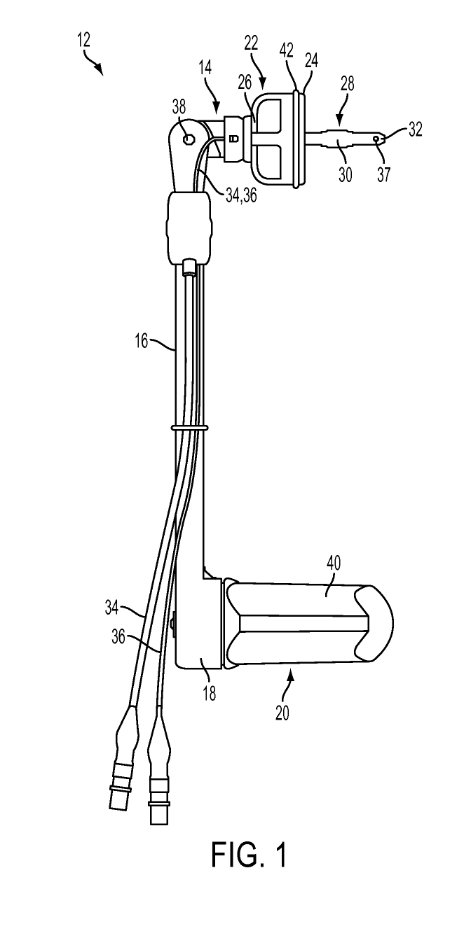

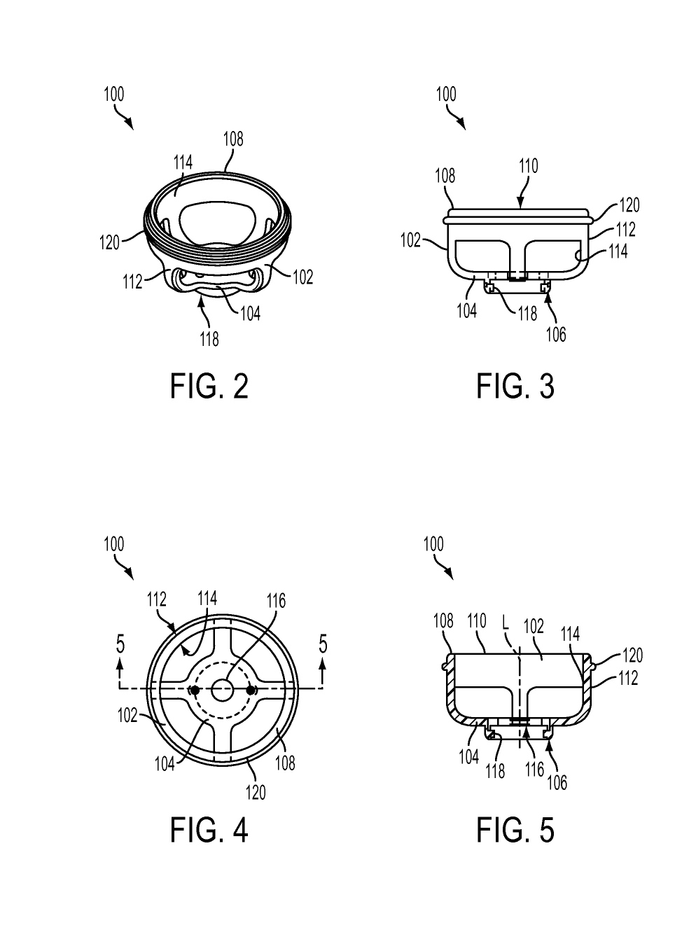

FIGS. 2 through 5 illustrate a cup 100 for use with a uterine manipulator, such as, by way of non-limiting example, the manipulator 12 of FIG. 1. The cup 100 may also be used with uterine manipulators of similar and dissimilar design, including, without limitation, those disclosed in U.S. Pat. No. 5,643,311, which issued on Jul. 1, 1997 to Smith et al., and U.S. Pat. No. 5,487,377, which issued on Jan. 30, 1996 to Smith et al., both of which are assigned to the assignee of the present disclosure and the disclosures of which are incorporated herein in their entireties by this reference. The cup 100 may include a cylindrical wall 102 extending between a base 104 located at a proximal end 106 of the cup 100 and a rim 108 located at a distal end 110 of the cup 100. The cylindrical wall 102 includes an outer surface 112 and an inner surface 114. The rim 108 is sized to surround, or at least substantially envelop, the anterior and posterior vaginal formix and may extend either continuously or non-continuously around the periphery of the distal end 110 of the cup 100. The rim 108 may be beveled and/or polished, as depicted, to reduce the amount of stress imparted on the formix by the cup 100. The base 104 is configured to abut the cervix and defines a central aperture 116 formed therein through which a longitudinal axis L of the cup 100 extends, as illustrated in FIGS. 4 and 5, respectively. The aperture 116 is configured to substantially align with the cervical os when the cup 100 engages the cervix. The aperture 116 has a diameter sufficient to allow extension therethrough of a tip section of a uterine manipulator, such as tip section 28 of the manipulator 12 of FIG. 1. As described previously, the tip section 28 may include a supple tip 32, a balloon 30 for inflating to engage the interior uterine wall, and a port 37 for injecting dye into the uterus. The tip section 28 may also include one or more tubes 34, 36 having lumens for respectively communicating fluid and dye to the balloon 30 and port 37.

Referring again to FIG. 2, the base 104 includes a mounting structure 118 for coupling the cup 100 to another component of a manipulator assembly, such as the rigid member 16 shown in FIG. 1. As shown in FIG. 2, the cup 100 may include a radially-extending circumferential protrusion 120 located on the outer surface 112 of the cylindrical wall 102 proximate the rim 108. The protrusion 120 may be beveled and/or polished to reduce the amount of stress imparted by the protrusion 120 to the uterus. As previously described, the protrusion 120 is configured to allow a physician to visually locate the rim 108 when the cervix is sufficiently engaged by the cup 100. In most embodiments, the cervix is sufficiently engaged by the cup 100 when the base 104 of the cup 100 abuts the cervix. In such a manner, the physician may form an incision using an electrosurgical or harmonic scalpel, as described above, in the vaginal formices proximate a portion of the protrusion 120.

A cup 100, as described above and in accordance with one or more embodiments of the disclosure, is formed from a substantially electrically insulating material that possesses a melting temperature sufficiently high enough to substantially withstand the intense thermal loading applied by a harmonic scalpel without substantially warping, burning, melting, producing jagged melt edges, emitting undesirable gasses therefrom, fracturing into or dissociating harmful particulate fragments, or fracturing the scalpel.

At least nine potential materials for cup 100 were tested by the inventors: (1) high-density polyethylene (HDPE); (2) polycarbonate; (3) polytherimide (PEI), specifically, ULTEM° brand PEI; (4) thirty percent glass-filled polybutylene terephthalate (30% GF PBT), specifically, VALOX° brand 30% GF PBT; (5) polyphenylene oxide/polystyrene alloy (PPO/OS), specifically, NORYL° brand PPO/OS; (6) thirty percent glass-filled polyphthalamide (30% GF PPA), specifically AMODEL° brand A-1133 HS NT PPA; (7) polyphthalamide (PPA) with no glass fill, specifically AMODEL° brand AT-1002 HS NT PPA; (8) polyetheretherketone (PEEK), specifically, PEEK-OPTIMA® brand PEEK; and (9) forty-five percent glass-filled polyphthalamide (45% GF PPA), specifically, AMODEL® brand A-1145 HS NT PPA. It is to be appreciated that the glass-filled percentage of certain of the above-referenced test materials (e.g., materials (4), (6) and (9)) represents a volume percentage of glass fill. The test results are presented herein in Tables 1 through 9. The material samples were repeatedly tested under exposure to both a harmonic scalpel and a simulated CO₂ laser scalpel against a variety of parameters, including the cut area and cut time. The harmonic scalpel used was an Ethicon brand, ULTRACISION® model harmonic scalpel. The simulated CO₂ laser scalpel used was a CO₂ laser welder. Some of the material samples were provided in the form of a sample plaque, while other material samples were provided in the form of a commercially available colpotomy cup. For each test run, the normalized cut time was calculated to reconcile the differences in the types of sample materials tested. Tests were not conducted with an electrosurgical scalpel, as the materials in question each comprise a proven electrically insulative material. For each tested material, the resulting behavioral characteristics were observed during and after exposure to each of the laser scalpel and the harmonic scalpel. It is to be appreciated that it is generally desirable for the test material to withstand longer durations of exposure to the harmonic and electrosurgical scalpels than would normally be experienced during a surgical procedure before exhibiting deleterious characteristics, such as those described above. The greater the extent to which the material withstands exposure to the cutting devices, the longer the physician may take to make a careful, accurate incision in the uterine wall.

Table 1 provides the test result data for high-density polyethylene (HDPE). Two test runs were conducted on the HDPE, with an average cut area of 0.179 square inches (in²) and an average cut time of 25.85 seconds (s), resulting in a normalized cutting time of 144.413 seconds per square inch (s/in²). It was observed that when the HDPE test material was exposed to the harmonic

TABLE 1
High-Density Polyethylene (HDPE)

| | Area Cut | Cut Time | Normalized |
|---|---|---|---|
| Test No. | (in²) | (s) | Time (s/in²) |
| 1 | 0.179 | 26.8 | 149.721 |
| 2 | 0.179 | 24.9 | 139.106 |
| Mean | 0.179 | 25.85 | 144.413 |

scalpel, the HDPE exhibited an unsatisfactory “stringy” quality. When exposed to the laser scalpel, the HDPE (a translucent material) was not affected, and it was observed that the laser beam passed through the material.

Table 2 provides the test result data for polycarbonate. Five test runs were conducted on the polycarbonate, with an average cut area of 0.026 square inches (in²) and an average cut time of 4.60 seconds (s), resulting in a normalized cutting time of 176.846 seconds per square inch (s/in²). It was observed that when the polycarbonate test material was exposed to the harmonic scalpel, unsatisfactory jagged melt edges formed in the polycarbonate, although not as severe as those formed in the polyetherimide (PEI), discussed below. When exposed to the laser

TABLE 2
Polycarbonate

| | Area Cut | Cut Time | Normalized |
|---|---|---|---|
| Test No. | (in²) | (s) | Time (s/in²) |
| 1 | 0.026 | 4.53 | 174.231 |
| 2 | 0.026 | 4.78 | 183.846 |
| 3 | 0.026 | 4.81 | 185.000 |
| 4 | 0.026 | 4.34 | 166.923 |
| 5 | 0.026 | 4.53 | 174.231 |
| Mean | 0.026 | 4.60 | 176.846 |

scalpel, the polycarbonate exhibited limited melting. The color of the sample made it difficult to determine whether any burning of the polycarbonate occurred from exposure to the laser scalpel.

Table 3 provides the test result data for polyetherimide (PEI). Five test runs were conducted on the PEI, with an average cut area of 0.05 square inches (in²) and an average cut time of 11.00 seconds (s), resulting in a normalized cutting time of 220.000 seconds per square inch (s/in²). It was observed that when the PEI test material was exposed to the harmonic scalpel, severe jagged melt edges formed in the PEI. Additionally, visible particulates, comparably sized to grains

TABLE 3
Polyetherimide (PEI)

| | Area Cut | Cut Time | Normalized |
|---|---|---|---|
| Test No. | (in²) | (s) | Time (s/in²) |
| 1 | 0.050 | 11.91 | 238.200 |
| 2 | 0.050 | 10.85 | 217.000 |
| 3 | 0.050 | 11.09 | 221.800 |
| 4 | 0.050 | 10.87 | 217.400 |
| 5 | 0.050 | 10.28 | 205.600 |
| Mean | 0.050 | 11.00 | 220.000 |

of sand, were observed as a result of the cut. When exposed to the laser scalpel, it was observed that the laser beam did not penetrate the PEI to a significant extent, yet burns were readily observed (more severe than on other tested materials).

Table 4 provides the test result data for a thirty percent glass-filled polybutylene terephthalate (30% GF PBT). Five test runs were conducted on the 30% GF PBT, with an average cut area of 0.024 square inches (in²) and an average cut time of 5.45 seconds (s), resulting in a normalized cutting time of 227.167 seconds per square inch (s/in²). While no particulates were observed when the 30% GF PBT test material was exposed to the harmonic scalpel, burning

TABLE 4
Polybutylene Terephthalate (PBT)

| | Area Cut | Cut Time | Normalized |
|---|---|---|---|
| Test No. | (in²) | (s) | Time (s/in²) |
| 1 | 0.024 | 5.88 | 245.000 |
| 2 | 0.024 | 5.63 | 234.583 |
| 3 | 0.024 | 5.34 | 222.500 |
| 4 | 0.024 | 5.25 | 218.750 |
| 5 | 0.024 | 5.16 | 215.000 |
| Mean | 0.024 | 5.45 | 227.167 |

occurred, in addition to the unfavorable release of gas. When exposed to the laser scalpel, rapid burning was observed, even when faster cuts were made by the laser, despite the fact that the laser beam did not penetrate as deeply into the material as compared with other tested materials.

Table 5 provides the test result data for a polyphenylene oxide/polystyrene alloy (PPO/OS). Five test runs were conducted on the PPO/OS, with an average cut area of 0.041 square inches (in²) and an average cut time of 12.23 seconds (s), resulting in a normalized cutting time of 298.390 seconds per square inch (s/in²). Jagged melt edges were formed when the PPO/OS test material was exposed to the harmonic scalpel, although less severe than those formed in the polyetherimide (PEI), referenced above. In addition, particulates were observed resulting from exposure to the harmonic scalpel, though smaller than the particulates observed in the PEI. When exposed to the laser scalpel, the PPO/OS melted readily and deeply. It was also observed that the

TABLE 5
Polyphenylene Oxide (PPO/OS)

| | Area Cut | Cut Time | Normalized |
|---|---|---|---|
| Test No. | (in²) | (s) | Time (s/in²) |
| 1 | 0.041 | 11.62 | 283.415 |
| 2 | 0.041 | 12.43 | 303.171 |
| 3 | 0.041 | 12.63 | 308.049 |
| 4 | 0.041 | 12.59 | 307.073 |
| 5 | 0.041 | 11.9 | 290.244 |
| Mean | 0.041 | 12.23 | 298.390 |

PPO/OS allowed burning when the laser beam was allowed to dwell at a single location, though charring was minimal and the laser beam had to dwell on the same location significantly longer to burn the PPO/OS than to burn other tested materials.

Table 6 provides the test result data for a thirty percent glass-filled polyphthalamide (30% GF PPA). Five test runs were conducted on the 30% GF PPA, with an average cut area of 0.027 square inches (in²) and an average cut time of 8.07 seconds (s), resulting in a normalized cutting time of 304.765 seconds per square inch (s/in²). It was observed that when the 30% GF PPA test material was exposed to the harmonic scalpel, burning of the material and smoke emanating therefrom were observed, in addition to a strong smell of burning plastic during

TABLE 6
Polyphthalamide (PPA) - 30% glass-filled

| | Area Cut | Cut Time | Normalized |
|---|---|---|---|
| Test No. | (in²) | (s) | Time (s/in²) |
| 1 | 0.030 | 8.63 | 287.667 |
| 2 | 0.028 | 8.69 | 310.357 |
| 3 | 0.030 | 8.35 | 278.333 |
| 4 | 0.027 | 8.06 | 298.519 |
| 5 | 0.019 | 6.63 | 348.947 |
| Mean | 0.027 | 8.07 | 304.765 |

the cutting tests. However, a minimal amount of particulates resulted from exposing the material to the harmonic scalpel. Furthermore, after a certain cut time, the harmonic scalpel would autonomously cease cutting the test material, apparently detecting excessive cutting resistance, and ceasing prior to producing more than the minimal amount of particulates previously described. When exposed to the laser scalpel, it was observed that the laser barely scratched the material when the laser was in motion, but burns were observed when the laser beam was allowed to dwell on a single location of the material. Overall, the 30% GF PPA was observed to be suitable for use with both harmonic and laser scalpels.

Table 7 provides the test result data for a polyphthalamide (PPA) with no glass fill. Five test runs were conducted on the PPA, with an average cut area of 0.023 square inches (in²) and an average cut time of 7.76 seconds (s), resulting in a normalized cutting time of 332.618 seconds per square inch (s/in²). The results observed when the PPA was exposed to the harmonic scalpel were similar to those observed for the thirty percent glass-filled polyphthalamide (30% GF PPA), discussed above. However, when exposed to the laser scalpel, it was observed that the PPA melted much more readily and to a greater depth than the 30% GF PPA, although the PPA did not

TABLE 7
Polyphthalamide (PPA) - no glass fill

| | Area Cut | Cut Time | Normalized |
|---|---|---|---|
| Test No. | (in²) | (s) | Time (s/in²) |
| 1 | 0.022 | 8 | 363.636 |
| 2 | 0.018 | 6.18 | 343.333 |
| 3 | 0.025 | 7.5 | 300.000 |
| 4 | 0.025 | 7.44 | 297.600 |
| 5 | 0.027 | 9.68 | 358.519 |
| Mean | 0.023 | 7.76 | 332.618 |

exhibit any burns, even when the laser beam was allowed to dwell on a single location of the PPA test material. Overall, the unfilled PPA was observed to be suitable for use with both harmonic and laser scalpels.

Table 8 provides the test result data for polyetheretherketone (PEEK). Five test runs were conducted on the PEEK, with an average cut area of 0.017 square inches (in²) and an average cut time of 7.23 seconds (s), resulting in a normalized cutting time of 415.382 seconds per square inch (s/in²). When the PEEK test material was exposed to the harmonic scalpel, it was observed that jagged melt edges were formed in the material, though not as severe as those that formed in the polyetherimide (PEI), discussed above. Particulates also resulted from exposing the PEEK to the harmonic scalpel, though the particulates were finer than those observed from the tests conducted on the PEI. Furthermore, after a certain cut time, the harmonic scalpel would autonomously cease cutting the test material, apparently detecting excessive resistance to cutting, and ceasing prior to producing more than the jagged melt edges and

TABLE 8
Polyetheretherketone (PEEK)

| | Area Cut | Cut Time | Normalized |
|---|---|---|---|
| Test No. | (in²) | (s) | Time (s/in²) |
| 1 | 0.020 | 8.57 | 428.500 |
| 2 | 0.020 | 8.34 | 417.000 |
| 3 | 0.015 | 6.44 | 429.333 |
| 4 | 0.015 | 6.19 | 412.667 |
| 5 | 0.017 | 6.62 | 389.412 |
| Mean | 0.017 | 7.23 | 415.382 |

particulates previously described. When exposed to the laser scalpel, it was observed that the laser barely scratched the PEEK when the laser was in motion, but burns were observed when the laser beam was allowed to dwell on a single location of the material, although the time required to produce these burns was longer than any of the samples involving polyphthalamide (PPA). Overall, the PEEK was observed to be suitable for use with both harmonic and laser scalpels.

Table 9 provides the test result data for a forty-five percent glass-filled polyphthalamide (45% GF PPA). Five test runs were conducted on the 45% GF PPA, with an average cut area of 0.0056 square inches (in²) and an average cut time of 3.13 seconds (s), resulting in a normalized cutting time of 558.772 seconds per square inch (s/in²). The results observed when the 45% GF PPA was exposed to the harmonic scalpel were similar to those observed for both the

TABLE 9
Polyphthalamide (PPA) - 45% glass-filled

| | Area Cut | Cut Time | Normalized |
|---|---|---|---|
| Test No. | (in²) | (s) | Time (s/in²) |
| 1 | 0.0056 | 2.78 | 496.429 |
| 2 | 0.0054 | 3 | 555.556 |
| 3 | 0.0056 | 3.16 | 564.286 |
| 4 | 0.0060 | 3.31 | 551.667 |
| 5 | 0.0054 | 3.38 | 625.926 |
| Mean | 0.0056 | 3.13 | 558.772 |

thirty percent glass-filled polyphthalamide (30% GF PPA) and the polyphthalamide (PPA) with no glass fill, discussed above. When exposed to the laser scalpel, it was observed that the 45% GF PPA was barely scratched by the laser beam when the laser was in motion, but burns were observed when the laser beam was allowed to dwell on a single location of the material. Overall, the 45% GF PPA was observed to be suitable for use with both harmonic and laser scalpels.

Based on the test results described above, the inventors have discovered, surprisingly and unexpectedly, that seemingly similar polymeric materials based on known material specifications behaved in remarkably different manners when exposed to the stimuli of a harmonic scalpel and a CO₂ laser scalpel. As a consequence of this discovery, it was established by the inventors that a colpotomy cup formed from polyetheretherketone (PEEK), or any of the polyphthalamide materials tested, including thirty percent glass-filled polyphthalamide (30% GF PPA); polyphthalamide (PPA) with no glass fill; and forty-five percent glass-filled polyphthalamide (45% GF PPA), is suitable for use with electrosurgical scalpels, laser scalpels and harmonic scalpels because these electrically insulating materials were observed to withstand substantial exposure to harmonic and laser types of scalpels without substantially warping, burning, melting, producing jagged melt edges, emitting undesirable gasses therefrom, fracturing into harmful particles or dissociating particulate fragments, or fracturing the scalpel in ways that may be harmful to the patient, the cup, or the scalpel.

As a further consequence of this discovery, it is believed that, when used with a harmonic scalpel, a colpotomy cup formed from polyetheretherketone (PEEK), or any of the polyphthalamide materials tested, including thirty percent glass-filled polyphthalamide (30% GF PPA); polyphthalamide (PPA) with no glass fill; and forty-five percent glass-filled polyphthalamide (45% GF PPA), will cause the contact with the harmonic scalpel to cause the harmonic scalpel to autonomously cease cutting before the cup substantially warps, burns, melts, produces jagged melt edges, emits undesirable gasses therefrom, fractures cup material into harmful particles or dissociates particulate fragments of the cup, or fractures the scalpel in ways that may be harmful to the patient, the cup, or the scalpel.

Accordingly, an entire colpotomy cup 100 (as shown in FIGS. 2 through 5), or any portion thereof likely to be contacted by a scalpel component (i.e., blade or laser beam), such as the rim 108, base 104, cylindrical wall 102, protrusion 120, and/or mounting structure 118, may be formed from any one of polyetheretherketone (PEEK); polyphthalamide (PPA) with no glass fill; thirty percent glass-filled polyphthalamide (30% GF PPA); forty-five percent glass-filled polyphthalamide (45% GF PPA); or any other type of polyphthalamide (PPA). It is to be appreciated that these materials are not required to be of a specific brand of materials. For example, in embodiments where all or part of the cup 100 comprises PPA, the PPA may be any one of (1) generic PPA; (2) AMODEL® PPA, manufactured by Solvay Advanced Polymers, LLC, located in Alpharetta, Ga.; (3) ZYTEL® PPA, manufactured by DuPont de Nemours and Company Corp., headquartered in Wilmington, Del.; or (4) any other brand of PPA.

A colpotomy cup according to embodiments described herein may be fabricated for use with a uterine manipulator in a colpotomy procedure or other medical procedure involving the female reproductive organs. For example, a cup-like structure for engaging a cervix, similar to the cup 100 illustrated in FIGS. 2 through 5, may be made by forming a rim 108 on the cup-like structure 100 configured to surround at least a portion of the anterior and posterior vaginal formix and forming a base 104 on the cup-like structure 100 defining an aperture 116 configured for one or more tubular members, such as tubes 34, 36 of FIG. 1, to extend therethrough; and forming the rim 108 and the base 104 of the cup-like structure 100 from a polyphthalamide (PPA) and/or a polyetheretherketone (PEEK) material.

The embodiments disclosed herein enable a single colpotomy cup to be used with electrosurgical scalpels, laser scalpels and harmonic scalpels.

While certain illustrative embodiments have been described in connection with the figures, those of ordinary skill in the art will recognize and appreciate that embodiments of the disclosure are not limited to those embodiments explicitly shown and described herein. Rather, many additions, deletions, and modifications to the embodiments described herein may be made without departing from the scope of the invention as hereinafter claimed, including legal equivalents. In addition, features from one disclosed embodiment may be combined with features of another disclosed embodiment while still being encompassed within the scope of the invention as contemplated by the inventors.

What is claimed is:

1. An intrauterine manipulator assembly, comprising a distal portion in communication with a handle, the distal portion comprising:
a balloon for engaging the inside of a uterine wall of a patient; and
a cup-like structure for engaging a cervix of the patient, the cup-like structure including a rim, and a base defining an aperture through which one or more tubular members extend, the base located proximate the balloon, the cup-like structure consisting essentially of one of a polyphthalamide (PPA) and a polyetheretherketone (PEEK).

2. The intrauterine manipulator assembly of claim 1, wherein the cup-like structure consists essentially of about a 30 percent glass-filled polyphthalamide (PPA).

3. The intrauterine manipulator assembly of claim 1, wherein the cup-like structure consists essentially of about a 45 percent glass-filled polyphthalamide (PPA).

4. The intrauterine manipulator assembly of claim 1, wherein the cup-like structure consists essentially of polyphthalamide (PPA) having substantially no glass fill therein.

5. The intrauterine manipulator assembly of claim 1, wherein the cup-like structure consists essentially of polyetheretherketone (PEEK).

6. A cup-like structure for engaging a cervix of a patient, comprising:
a rim configured to surround at least a portion of an anterior and posterior vaginal formix of the patient; and
a base defining an aperture therein, wherein at least one of the rim and the base consists essentially of one of a polyphthalamide and a polyetheretherketone (PEEK).

7. The cup-like structure of claim 6, wherein the cup-like structure substantially does not disassociate particulate fragments when contacted with a harmonic cutting device.

8. The cup-like structure of claim 6, wherein the cup-like structure substantially does not melt or burn when exposed to a CO₂ laser cutting device.

9. The cup-like structure of claim 6, wherein the material of the at least one of the rim and the base exhibits a melting temperature at least substantially equivalent to the higher of a temperature of radiation emitted from a CO₂ laser cutting device and a temperature produced in the material of the one of the rim and the base by contact with a harmonic cutting device.

10. The cup-like structure of claim 8, wherein the cup-like structure substantially does not form jagged melt edges when contacted with a harmonic cutting device.

11. The cup-like structure of claim 10, wherein the cup-like structure substantially does not emit smoke or gas when contacted with a harmonic cutting device.

12. The cup-like structure of claim 11, wherein the cup-like structure consists essentially of about a 30 percent glass-filled polyphthalamide.

13. The cup-like structure of claim 11, wherein the cup-like structure consists essentially of about a 45 percent glass-filled polyphthalamide.

14. The cup-like structure of claim 11, wherein the cup-like structure consists essentially of a polyphthalamide having substantially no glass fill therein.

15. The cup-like structure of claim 11, wherein the cup-like structure consists essentially of polyetheretherketone (PEEK).
